SEQUENCE LISTING (9 sequences)

SEQ ID NO: 1 - DNA, 29 bp, Artificial sequence
  synthetic sequence
ggttaattttgctcagagtatccagagtt

SEQ ID NO: 2 - DNA, 29 bp, Artificial sequence
  synthetic sequence
ggttaattttgctcagagtatccggagtt

SEQ ID NO: 3 - DNA, 26 bp, Artificial sequence
  synthetic sequence
gcaggcaagggatcgtttctcctctt

SEQ ID NO: 4 - DNA, 29 bp, Artificial sequence
  synthetic sequence
tccagatagagctaaaactgaagtuttcc

SEQ ID NO: 5 - DNA, 29 bp, Artificial sequence
  synthetic sequence
tccagatagagctaaaactgaagcuttcc

SEQ ID NO: 6 - DNA, 25 bp, Artificial sequence
  synthetic sequence
gcaaccagtatccccgcaaactaac

SEQ ID NO: 7 - DNA, 27 bp, Artificial sequence
  synthetic sequence
ccgtcattctctcagttcttatctctg

SEQ ID NO: 8 - DNA, 27 bp, Artificial sequence
  synthetic sequence
ccgtcattctctcagttcttacctctg

SEQ ID NO: 9 - DNA, 28 bp, Artificial sequence
  synthetic sequence
gccttctctgcacagttcattggctcct

(19) United States
(12) Patent Application Publication
(10) Pub. No.: US 2024/0053335 A1
(43) Pub. Date: Feb. 15, 2024

(54) Capillary Microsample Analysis Methods

(71) Applicants: The Board of Trustees of the Leland Stanford Junior University, Stanford, CA (US); Microdrop Inc., Saratoga, CA (US)

(72) Inventors: Gary Peltz, Redwood City, CA (US); Feng Hu, Saratoga, CA (US)

(21) Appl. No.: 18/268,932

(22) PCT Filed: Dec. 20, 2021
(86) PCT No.: PCT/US2021/064403

§ 371 Date: Jun. 21, 2023

Related U.S. Application Data
(60) Provisional application No. 63/129,239, filed on Dec. 22, 2020.

(51) Int. Cl. G01N 33/543; G01N 33/94; C12Q 1/6806; C12Q 1/6858

(52) U.S. Cl. CPC G01N 33/54393; G01N 33/948; G01N 33/9486; G01N 33/946; C12Q 1/6806; C12Q 1/6858

(57) ABSTRACT

Provided are methods of analyzing a capillary microsample obtained from a subject. In certain embodiments, the methods comprise assessing a test mixture for one or more capillary microsample analytes, wherein the test mixture comprises the capillary microsample diluted into a stabilizing buffer comprising isopropanol, ethanol, methanol, or a combination thereof. Such methods further comprise analyzing capillary microsample nucleic acids purified from the test mixture. According to some embodiments, analyzing the capillary microsample nucleic acids comprises genotyping the subject. Methods of determining one or more alleles of a subject are also provided, as are kits that find use in practicing the methods of the present disclosure.

### CROSS-REFERENCE TO RELATED APPLICATIONS

This application claims the benefit of U.S. Provisional Patent Application No. 63/129,239, filed Dec. 22, 2020, which application is incorporated herein by reference in its entirety.

### INTRODUCTION

Modern medicine requires a precise determination of the concentrations of many different analytes in bodily fluids. Opioid levels are measured in opioid dependent subjects to assess compliance and to prevent diversion. More broadly, drug levels are frequently measured to assess compliance with a prescribed regimen or for dosage adjustment to ensure that therapeutic (rather than ineffective or toxic) levels are obtained. Measuring drug levels is particularly important in infants or the elderly, where the optimal drug dose is often unknown, or for drugs with very narrow therapeutic ranges. Venipuncture is now required to obtain enough plasma (usually 5-10 ml) for most diagnostic tests. This procedure is particularly difficult for infants or the elderly, especially when repeated testing is required for therapeutic monitoring. Subjects must go to a laboratory with specially trained technicians, which creates a barrier for vulnerable populations (i.e., pregnant women, elderly, pediatric/neonatal). While blood can be placed on filter paper for some measurements, this introduces significant inaccuracy in quantitative measurements. In addition, utilization of pharmacogenetic (PGx) information could improve treatment outcome by optimizing drug or dose selection based upon an individual's genetic makeup. A convenient method for simultaneously obtaining drug levels and PGx information from the same microsample would dramatically increase our ability implement the ‘personalized medicine’ strategies that have become a focus of 21^{st} century healthcare. Current approaches do not permit accurate measurement of analyte levels and PGx information in small blood volumes. Moreover, existing methodologies for measuring drug levels suffer from subject privacy issues (e.g., samples cannot be taken at home) because sample donor identity cannot be determined/verified. The present disclosure addresses these and other deficiencies of the current methods for sample analysis.

### SUMMARY

Provided are methods of analyzing a capillary microsample obtained from a subject. In certain embodiments, the methods comprise assessing a test mixture for one or more capillary microsample analytes, wherein the test mixture comprises the capillary microsample diluted into a stabilizing buffer comprising isopropanol, ethanol, methanol, or a combination thereof. Such methods further comprise analyzing capillary microsample nucleic acids purified from the test mixture. According to some embodiments, analyzing the capillary microsample nucleic acids comprises genotyping the subject. Methods of determining one or more alleles of a subject are also provided, as are kits that find use in practicing the methods of the present disclosure.

### BRIEF DESCRIPTION OF THE FIGURES

FIG. 1 provides data demonstrating that genotypes can be determined according to the methods of the present disclosure. In this example, 8 μL capillary microsamples were diluted into an isopropanol-containing stabilizing buffer (producing “iso-CMS” samples). The alleles at 3 SNP sites in 12 iso-CMS samples were determined by allele-specific qPCR: UBAC2 rs1058083 (reference and alternative alleles are A/G); FZD3 rs10092491 (T/C); and UXS1 rs10185531 (T/C). The genotyping results are shown in three scatterplots. Each shape shows the two-channel fluorescence (normalized relative to background) for the reference (y-axis) and alternative (x-axis) alleles at each SNP. The particular shape indicates the genotyping call at each SNP: triangles, homozygous alternative alleles; circles, heterozygous; and squares, homozygous reference alleles. The twelve individuals can be distinguished based upon the allelic combinations determined for these 3 SNPs.

FIG. 2 provides data demonstrating that ondansetron concentrations measured by iso-CMS correspond with the plasma measurements in clinical subjects. Sixty plasma and iso-CMS samples were simultaneously obtained from 44 subjects receiving ondansetron, and drug concentrations were measured by LCMS analysis. The therapeutic [ONDS] are between 10 and 100 ng/ml, which is the region of the graph where many datapoints clustered (top panel). Therefore, to better present the ondansetron data, the region with [ONDS] between 15 and 280 ng/ml is shown separately (bottom panel). Each data point shows the measured drug concentration determined from the iso-CMS and plasma samples for each subject. The R² assessing the concordance between all iso-CMS and plasma measurements was 0.99, and the R² for samples with [ONDS]<300 ngl/ml was 0.95.

FIG. 3 provides data demonstrating that dexamethasone, gabapentin, and methadone concentrations measured by iso-CMS analysis correspond with the plasma measurements in clinical subjects. Plasma and iso-CMS samples were simultaneously obtained from 29, 15 or 22 subjects after they received dexamethasone, gabapentin or methadone, respectively. The drug concentrations were measured by LCMS analysis. Each data point shows the measured drug concentration in the iso-CMS and plasma samples for each subject. The R² assessing the concordance between the iso-CMS and plasma measurements for each drug are shown.

### DETAILED DESCRIPTION

Before the methods and kits of the present disclosure are described in greater detail, it is to be understood that the methods and kits are not limited to particular embodiments described, as such may, of course, vary. It is also to be understood that the terminology used herein is for the purpose of describing particular embodiments only, and is not intended to be limiting, since the scope of the methods and kits will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the methods and kits. The upper and lower limits of these smaller ranges may independently be included in the smaller ranges and are also encompassed within the methods and kits, subject to any specifically excluded limit in the stated range. Where the stated range includes one or both of the limits, ranges excluding either or both of those included limits are also included in the methods and kits.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which the methods and kits belong. Although any methods and kits similar or equivalent to those described herein can also be used in the practice or testing of the methods and kits, representative illustrative methods and kits are now described.

All publications and patents cited in this specification are herein incorporated by reference as if each individual publication or patent were specifically and individually indicated to be incorporated by reference and are incorporated herein by reference to disclose and describe the materials and/or methods in connection with which the publications are cited. The citation of any publication is for its disclosure prior to the filing date and should not be construed as an admission that the present methods and kits are not entitled to antedate such publication, as the date of publication provided may be different from the actual publication date which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the methods and kits, which are, for clarity, described in the context of separate embodiments, may also be provided in combination in a single embodiment. Conversely, various features of the methods and kits, which are, for brevity, described in the context of a single embodiment, may also be provided separately or in any suitable sub-combination. All combinations of the embodiments are specifically embraced by the present disclosure and are disclosed herein just as if each and every combination was individually and explicitly disclosed, to the extent that such combinations embrace operable processes and/or compositions. In addition, all sub-combinations listed in the embodiments describing such variables are also specifically embraced by the present methods and kits and are disclosed herein just as if each and every such sub-combination was individually and explicitly disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present methods. Any recited method can be carried out in the order of events recited or in any other order that is logically possible.

### Methods

The present disclosure provides methods of analyzing a capillary microsample obtained from a subject. In certain embodiments, the methods comprise assessing a test mixture for one or more capillary microsample analytes (e.g., one or more drugs of abuse, one or more therapeutic drugs or analytes thereof, or the like), wherein the test mixture comprises the capillary microsample diluted into a stabilizing buffer comprising isopropanol, ethanol, methanol, or a combination thereof. Such methods further comprise analyzing capillary microsample nucleic acids purified from the test mixture. According to some embodiments, analyzing the capillary microsample nucleic acids comprises genotyping the subject, e.g., to determine/confirm the identity of the subject, determine one or more alleles relevant to a therapy being administered to the subject, and/or the like.

The present methods provide a number of advantages over existing sample analysis approaches. For example, in certain embodiments, provided are convenient methods for simultaneously obtaining drug levels and pharmacogenetic (PGx) information from the same microsample, thereby facilitating “personalized medicine” strategies that have become a focus of 21^{st} century healthcare. Also by way of example, according to some embodiments, analyte (e.g., drug) analysis and genotyping are performed from the same capillary microsample, enabling combined capillary microsample analysis and sample donor identification which is advantageous in a variety of settings, including screening for drugs of abuse (sometimes referred to herein as “illicit drugs”) as well as ensuring compliance with a prescribed therapeutic regimen.

Regarding screening for drugs of abuse (e.g., opioids), urine drug testing (UDT) is the major surveillance method. Although UDT is a commonly used surveillance method, it has substantial problems which are addressed by the methods of the present disclosure. For example, since urine is collected in private, there are many ways to dilute a sample or to substitute a clean urine specimen. Multiple types of prosthetic penises are specifically sold to confound UDT which produce a realistic urine stream and have reservoirs that are strapped to the body (or heating units) to maintain the temperature of the stored “clean” urine. Second, a urine sample can be altered by adding agents that interfere with immunoassays used for opioid detection, which include household products (bleach, vinegar, detergent) or over the counter medications. According to some embodiments of the methods of the present disclosure, genotyping is performed on the same capillary microsample used for the illicit drug level measurement (or in other embodiments, therapeutic drug level measurement), enabling subsequent samples to be collected in many locations since donor identity could be confirmed by comparison of the genotyping results with those from prior samples in which the subject's identity was confirmed. Proof of concept that capillary microsamples diluted into stabilizing buffers may be genotyped to confirm sample donor identity is provided in the Experimental section below. More generally, the results therein demonstrate that identifying, diagnostic and/or prognostic allelic information can be obtained using capillary microsamples prepared according to the methods of the present disclosure, which samples can additionally be used for measurement of blood analyte levels, e.g., illicit drugs, therapeutic drugs or metabolites thereof, performance enhancing drugs, and/or the like. Further details regarding the methods of the present disclosure will now be provided.

Capillary Microsample Nucleic Acid Analysis

As noted above, the methods of the present disclosure comprise analyzing capillary microsample nucleic acids purified from a test mixture comprising the capillary microsample diluted into a stabilizing buffer comprising isopropanol, ethanol, methanol, or a combination thereof. The term “nucleic acid” and “polynucleotide” are used interchangeably herein to describe a polymer of any length, e.g., greater than about 2 bases, greater than about 10 bases, greater than about 100 bases, greater than about 500 bases, greater than 1000 bases, greater than 10,000 bases, greater than 100,000 bases, greater than about 1,000,000, up to about 10¹⁰ or more bases (e.g., a chromosome) composed of nucleotides, e.g., deoxyribonucleotides or ribonucleotides. Naturally-occurring nucleotides include guanine, cytosine, adenine, thymine, uracil (G, C, A, T and U respectively). DNA and RNA have a deoxyribose and ribose sugar backbones, respectively. By “capillary microsample nucleic acids” is meant one or more types of nucleic acids present in the blood capillary microsample obtained from the subject. In certain embodiments, the analysis comprises genomic DNA analysis, RNA analysis, or both genomic DNA analysis and RNA analysis.

According to some embodiments, the methods comprise analyzing cell-free nucleic acids present in the microcapillary sample, e.g., cell-free DNA, cell-free RNA, or both. The term “cell-free nucleic acid” as used herein can refer to nucleic acid isolated from a source having substantially no cells. Cell-free nucleic acid may be referred to as “extracellular” nucleic acid, “circulating cell-free” nucleic acid (e.g., CCF fragments, ccf DNA) and/or “cell-free circulating” nucleic acid. Cell-free nucleic acid can be present in and obtained from blood (e.g., from the blood of an animal, from the blood of a human subject). Cell-free nucleic acid often includes no detectable cells and may contain cellular elements or cellular remnants. Without being limited by theory, cell-free nucleic acid may be a product of cell apoptosis and cell breakdown, which provides basis for cell-free nucleic acid often having a series of lengths across a spectrum (e.g., a “ladder”).

Cell-free nucleic acid can include different nucleic acid species, and therefore is referred to herein as “heterogeneous” in certain embodiments. For example, a capillary microsample from a subject having cancer can include nucleic acid from cancer cells (e.g., tumor, neoplasia) and nucleic acid from non-cancer cells. In another example, a capillary microsample from a pregnant female can include maternal nucleic acid and fetal nucleic acid. In another example, a sample from a subject having an infection or infectious disease can include host nucleic acid and nucleic acid from the infectious agent (e.g., bacteria, fungus, protozoa). In another example, a capillary microsample from a subject having received a transplant can include host nucleic acid and nucleic acid from the donor organ or tissue. In some instances, cancer, fetal, infectious agent, or transplant nucleic acid sometimes is about 5% to about 50% of the overall nucleic acid (e.g., about 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or 49% of the total nucleic acid is cancer, fetal, infectious agent, or transplant nucleic acid).

In certain embodiments, the methods comprise analyzing circulating tumor DNA (ctDNA) present in the microcapillary sample. Such methods find use, e.g., in monitoring the efficacy of a cancer therapy being administered to the subject, assessing the subject for the presence and/or recurrence of cancer, and/or the like, based on ctDNA present in one or more microcapillary samples obtained from the subject, e.g., microcapillary samples serially obtained from the subject. ctDNA is found in the bloodstream and refers to DNA that comes from cancerous cells and tumors. Most DNA is inside a cell's nucleus. As a tumor grows, cells die and are replaced by new ones. The dead cells get broken down and their contents, including DNA, are released into the bloodstream. ctDNA are small pieces of DNA, usually comprising fewer than 200 nucleotides in length.

“Tumor”, as used herein, refers to all neoplastic cell growth and proliferation, whether malignant or benign, and all pre-cancerous and cancerous cells and tissues. The terms “cancer” and “cancerous” refer to or describe the physiological condition in mammals that is typically characterized by unregulated cell growth/proliferation. Examples of cancer include but are not limited to, carcinoma, lymphoma, blastoma, sarcoma, and leukemia. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, prostate cancer, vulval cancer, thyroid cancer, hepatic carcinoma, various types of head and neck cancer, and the like.

RNAs that may be analyzed according to the present methods include, but are not limited to, messenger RNA (mRNA), microRNA (miRNA), small interfering RNA (siRNA), transacting small interfering RNA (ta-siRNA), natural small interfering RNA (nat-siRNA), ribosomal RNA (rRNA), transfer RNA (tRNA), small nucleolar RNA (snoRNA), small nuclear RNA (snRNA), long non-coding RNA (lncRNA), non-coding RNA (ncRNA), transfer-messenger RNA (tmRNA), precursor messenger RNA (pre-mRNA), small Cajal body-specific RNA (scaRNA), piwi-interacting RNA (piRNA), small temporal RNA (stRNA), signal recognition RNA, telomere RNA, a ribozyme, or any combination of RNA types thereof or subtypes thereof. In certain embodiments, when the analysis includes RNA analysis, cDNAs produced by reverse-transcribing the RNA(s) of interest are analyzed. Approaches and kits for reverse transcribing RNAs of interest are known and include, e.g., the QuantiTect® reverse transcription kit (Qiagen, Germantown, MD).

The capillary microsample nucleic acids are purified from the test mixture. By “purified” in this context is meant the nucleic acids which are directly analyzed are substantially free of the stabilizing buffer such that the solution in which the purified nucleic acids are present comprises less than 20%, less than 10%, less than 5%, less than 1%, or less than 0.1% of the stabilizing buffer. Numerous suitable approaches and kits for purifying the capillary microsample nucleic acids from the test mixture are available. For example, the capillary microsample nucleic acids may be precipitated out of the test mixture and resuspended in a buffer (e.g., TE (Tris-EDTA) or the like) compatible with the analysis of interest (e.g., PCR, sequencing, etc.). According to some embodiments, a solid phase extraction/purification method, such as spin column-based nucleic acid purification, is employed. Kits for spin column-based nucleic acid purification are readily available and include the AllPrep™ DNA/RNA Mini Kit (Qiagen), Nucleospin™ DNA/RNA isolation kit (Takara), and the like.

A wide variety of nucleic acid analysis approaches may be employed to analyze the capillary microsample nucleic acids purified from the test mixture. In some embodiments, analyzing the capillary microsample nucleic acids comprises amplifying the capillary microsample nucleic acids, e.g., by polymerase chain reaction (PCR) amplification involving template denaturation, primer annealing and primer extension. The initial step denatures the target DNA by heating it to 94° C. or higher for 15 seconds to 2 minutes. In the denaturation process, the two intertwined strands of DNA separate from one another, producing the necessary single-stranded DNA template for replication by the thermostable DNA polymerase. In the next step of a cycle, the temperature is reduced to approximately 40-60° C. At this temperature, the oligonucleotide primers can form stable associations (anneal) with the denatured target DNA and serve as primers for the DNA polymerase. This step lasts approximately 15-60 seconds. Finally, the synthesis of new DNA begins as the reaction temperature is raised to the optimum for the DNA polymerase. For most thermostable DNA polymerases (e.g., Taq polymerase), this temperature is in the range of 70-74° C. The extension step lasts approximately 1-2 minutes. The next cycle begins with a return to the denaturation temperature. Each step of the cycle should be optimized for each template and primer pair combination. If the temperature during the annealing and extension steps are similar, these two steps can be combined into a single step in which both primer annealing and extension take place. After 20-40 cycles, the amplified product may be analyzed for size, quantity, sequence, etc., or used in further experimental procedures.

Suitable annealing conditions for PCR will depend upon the target to be amplified and the primers used for amplification such that specific hybridization is achieved. Whether specific hybridization occurs is determined by such factors as the degree of complementarity between the primers and the primer binding regions of the target nucleic acid, the length thereof, and the temperature at which the hybridization occurs, which may be informed by the melting temperatures (T_{M}) of the relevant portions of the primers and primer binding regions. The melting temperature refers to the temperature at which half of the primers remain hybridized and half of the primers dissociate into single strands. The Tm of a duplex may be experimentally determined or predicted using the following formula Tm=81.5+16.6(log 10[Na⁺])+0.41 (fraction G+C)−(600/N), where N is the chain length and [Na⁺] is less than 1 M. See Sambrook and Russell (2001; Molecular Cloning: A Laboratory Manual, 3rd ed., Cold Spring Harbor Press, Cold Spring Harbor N.Y., Ch. 10). Other more advanced models that depend on various parameters may also be used to predict Tm of primer/primer binding region duplexes depending on various hybridization conditions. Approaches for achieving specific nucleic acid hybridization may be found in, e.g., Tijssen, Laboratory Techniques in Biochemistry and Molecular Biology-Hybridization with Nucleic Acid Probes, part I, chapter 2, “Overview of principles of hybridization and the strategy of nucleic acid probe assays,” Elsevier (1993).

In certain embodiments, when analyzing the capillary microsample nucleic acids comprises amplifying the capillary microsample nucleic acids, the analysis comprises quantitative polymerase chain reaction (qPCR—also referred to as “real-time PCR” or “quantitative real-time PCR”). qPCR is a PCR-based technique that couples amplification of a target DNA sequence with quantification of the concentration of that DNA species in the reaction. This method enables calculation of the starting template concentration and is therefore a frequently used analytical tool in allelic discrimination, SNP detection, evaluating DNA copy number, viral load, etc. Reagents and kits for qPCR-base genotyping and other applications are available and include, e.g., the TaqPath™ ProAmp™ Master Mix (ThermoFisher), MeltDoctor™ HRM Master Mix (ThermoFisher), QuantiTect SYBR® Green PCR Kits (Qiagen), QuantiTect Multiplex PCR Kits (Qiagen), and the like. Instruments for performing real-time PCR are available and include the CFX Opus Real-Time PCR System (Bio-Rad), CFX Connect Real-Time PCR System (Bio-Rad), the QuantStudio™ real-time PCR systems (Applied Biosystems), and the like. Detailed protocols and primer design considerations are provided with such reagents, kits and systems.

According to some embodiments, analyzing the capillary microsample nucleic acids comprises sequencing the capillary microsample nucleic acids. Sequencing platforms that may be employed to sequence the capillary microsample nucleic acids are available and include a sequencing platform provided by Illumina® (e.g., the HiSeq™, NextSeq™, MiSeq™ and/or NovaSeq™ sequencing systems); Oxford Nanopore™ Technologies (e.g., a SmidgION, MinION, GridION, or PromethION nanopore-based sequencing system), Ion Torrent™ (e.g., the Ion PGM™ and/or Ion Proton™ sequencing systems); Pacific Biosciences (e.g., a Sequel II ZMW-based sequencing system); Life Technologies™ (e.g., a SOLiD sequencing system); Roche (e.g., the 454 GS FLX+ and/or GS Junior sequencing systems); or any other sequencing platform of interest. Detailed design considerations and protocols for preparing nucleic acids (e.g., any necessary adapter addition, etc.), conducting nucleic acid sequencing runs, and analyzing the resulting sequencing data are provided by the manufacturers of such systems.

In the Illumina platform, the sequencing process involves clonal amplification of adaptor-ligated DNA fragments on the surface of a glass slide. Bases are read using a cyclic reversible termination strategy, which sequences the template strand one nucleotide at a time through progressive rounds of base incorporation, washing, imaging, and cleavage. In this strategy, fluorescently labeled 3′-O-azidomethyl-dNTPs are used to pause the polymerization reaction, enabling removal of unincorporated bases and fluorescent imaging to determine the added nucleotide. Following scanning of the flow cell with a coupled-charge device (CCD) camera, the fluorescent moiety and the 3′ block are removed, and the process is repeated.

In zero mode waveguide (ZMW)-based sequence analysis (e.g., provided by Pacific Biosciences), the ZMW is a nanoscale-sized well that serves as an optical confinement that allows observation of individual polymerase molecules. As a result, nucleotide incorporation events provide observation of an incorporating nucleotide analog that is readily distinguishable from non-incorporated nucleotide analogs. For a description of ZMWs and their application in nucleic acid sequencing, see, e.g., U.S. Patent Application Publication No. 2003/0044781 and U.S. Pat. No. 6,917,726, each of which is incorporated herein by reference in its entirety for all purposes. See also Levene et al. (2003) “Zero-mode waveguides for single-molecule analysis at high concentrations” Science 299:682-686, Eid et al. (2009) “Real-time DNA sequencing from single polymerase molecules” Science 323:133-138, and U.S. Pat. Nos. 7,056,676, 7,056,661, 7,052,847, 7,033,764, and 7,907,800, the full disclosures of which are incorporated herein by reference in their entirety for all purposes.

In nanopore sequencing, the nanopore serves as a biosensor and provides the sole passage through which an ionic solution on the cis side of the membrane contacts the ionic solution on the trans side. A constant voltage bias (trans side positive) produces an ionic current through the nanopore and drives ssDNA or ssRNA in the cis chamber through the pore to the trans chamber. A processive enzyme (e.g., a helicase, polymerase, nuclease, or the like) may be bound to the polynucleotide such that its step-wise movement controls and ratchets the nucleotides through the small-diameter nanopore, nucleobase by nucleobase. Because the ionic conductivity through the nanopore is sensitive to the presence of the nucleobase's mass and its associated electrical field, the ionic current levels through the nanopore reveal the sequence of nucleobases in the translocating strand. A patch clamp, a voltage clamp, or the like, may be employed.

The methods of the present disclosure may include a variety of types of nucleic acid analyses of interest. In certain embodiments, analyzing the capillary microsample nucleic acids comprises determining one or more alleles of the subject. As used herein, an “allele” is one of two or more alternative forms of a genetic element occupying a specific region (or locus) on a chromosome. Non-limiting examples of genetic elements for which an allele may be determined include genes, polymorphisms, and the like. Accordingly, in certain embodiments, the one or more alleles comprises one or more polymorphic alleles. Non-limiting examples of polymorphic alleles that may be determined include one or more single nucleotide polymorphism (SNP) alleles, one or more variable number of tandem repeat (VNTR) alleles, one or more insertion/deletion (indel) alleles, one or more short tandem repeat (STR) alleles, or any combination thereof. When the methods include determining one or more SNP alleles, the one or more SNP alleles may comprise one or more minor allele frequency (MAF) SNP alleles. Such MAF SNPs are known and provided in the NCBI dbSNP database (www.ncbi.nlm.nih.gov/snp/). According to some embodiments, SNPs with MAFs ranging from 20% to 60% (e.g., 25% to 50%, such 25% to 48%) are selected since such SNPs have the most discriminatory power.

As noted above, determining one or more alleles of the subject may comprise genotyping the subject. Genotyping is the process of determining which genetic variant(s) (e.g., allele(s)) a subject possesses and may be used for a variety of purposes including determining/confirming the identity of the subject.

In certain embodiments, determining one or more alleles of the subject comprises determining an allele of one or more genes, i.e., one of two or more alternative forms of a gene of interest. Determining an allele of a gene of interest may comprise determining that the subject comprises a wild-type allele of the gene of interest, or a mutant allele of the gene of interest. Non-limiting examples of genes of interest include disease-associated genes, genes that provide pharmacogenetic (PGx) information (e.g., a gene whose product is involved in drug metabolism), and the like. In some embodiments, the one or more genes for which an allele is determined comprises one or more genes that encode a drug metabolizing enzyme, one or more genes that encode a drug receptor, and/or one or more genes that encode a drug transporter.

According to some embodiments, analyzing the capillary microsample nucleic acids comprises determining the presence or absence of a region of the Y-chromosome among genomic DNA present in the capillary microsample nucleic acids. Such an analysis may be performed to determine/confirm the sex of the subject from whom the capillary microsample was obtained. Non-limiting examples of male-specific genes on the Y-chromosome which may be used to assess the sex of the subject include SRY, AMEL, and others. See, e.g., Settin et al. (2008) Int J Health Sci (Qassim) 2:49-52.

Capillary Microsample Analyte Assessment

As noted above, the methods of the present disclosure comprise assessing the test mixture for one or more capillary microsample analytes. As used herein, a “capillary microsample analyte” is a non-nucleic acid analyte which may be present in the capillary microsample. The methods may comprise determining whether one or more capillary microsample analytes of interest are present in the test mixture (and, in turn, in the capillary microsample). In certain embodiments, assessing the test mixture for one or more capillary microsample analytes comprises determining the concentration of one or more capillary microsample analytes, if present, in the test mixture. Such a concentration may be converted to determine the concentration of the one or more capillary microsample analytes in the capillary microsample. Approaches that may be employed to assess the test mixture for one or more capillary microsample analytes include those described in PCT/US2018/058440 (which published as WO 2019/089746A1), the disclosure of which is incorporated herein by reference in its entirety for all purposes.

In certain embodiments, the one or more capillary microsample analytes comprise one or more small molecule analytes. By “small molecule” is meant a compound having a molecular weight of 1000 atomic mass units (amu) or less. In certain embodiments, the small molecule is 750 amu or less, 500 amu or less, 400 amu or less, 300 amu or less, or 200 amu or less. According to some embodiments, the small molecule is not made of repeating molecular units (monomers) such as are present in a polymer.

According to some embodiments, the one or more capillary microsample analytes comprise one or more polypeptide analytes. The terms “polypeptide”, “peptide”, or “protein” are used interchangeably herein to designate a linear series of amino acid residues connected one to the other by peptide bonds between the alpha-amino and carboxy groups of adjacent residues. The amino acids may include the 20 “standard” genetically encodable amino acids, amino acid analogs, or a combination thereof.

According to some embodiments, the one or more capillary microsample analytes comprises one or more drugs of abuse. Drugs of abuse include, but are not limited to, United States Drug Enforcement Administration (DEA) schedule I, schedule II, schedule Ill, schedule IV, and schedule V drugs. In certain embodiments, the one or more drugs of abuse comprises one or more of a narcotic, an opioid, a stimulant, a depressant, a hallucinogen, a cannabinoid, or any combination thereof. According to some embodiments, the one or more drugs of abuse comprises one or more opioids. In certain embodiments, the one or more opioids comprises oxycodone, hydrocodone, morphine, methadone, fentanyl, heroin, or any combination thereof. According to some embodiments, the one or more drugs of abuse comprises gabapentin.

In certain embodiments, the one or more capillary microsample analytes comprises one or more performance enhancing drugs. For example, the methods may include assessing the test mixture for one or more performance enhancing drugs selected from an anabolic steroid, androstenedione, human growth hormone (HGH), erythropoietin (EPO), a diuretic, creatine, a stimulant, or any combination thereof. According to some embodiments, when the methods comprise assessing the test mixture for one or more performance enhancing drugs, the subject from whom the capillary microsample was obtained is an upcoming, current, or former participant in an athletic contest. Examples of athletic contests include the Olympic games, a track meet, a cycle race (e.g., Tour de France), a football game, a baseball game, a basketball game, a soccer game, a triathlon, and/or the like. In some embodiments, the method is performed iteratively on test mixtures comprising capillary microsamples obtained serially from the subject prior to, during, and/or subsequent to the athletic contest. As with any of the methods of the present disclosure, the methods comprising assessment for performance enhancing drugs may be combined with genotyping to confirm the identity of the donor/subject.

According to some embodiments, the one or more capillary microsample analytes comprises a therapeutic drug being administered to the subject, or a metabolite thereof. Non-limiting examples of therapeutic drugs which may be assessed according to the methods of the present disclosure include therapeutic drugs for treatment of cancer, hypertension, epilepsy, hypercoagulation, or nausea.

As noted above, any of the methods of the present disclosure may be performed iteratively. For example, the methods may be performed iteratively on test mixtures comprising capillary microsamples obtained serially from the subject. The capillary microsamples may be obtained serially (and in turn, the methods may be performed iteratively) 2 or more, 3 or more, 4 or more, 5 or more, 6 or more, 7 or more, 8 or more, 9 or more, 10 or more, 15 or more, 20 or more, or 50 or more times on the same subject.

Approaches that may be employed for assessing the test mixture for one or more capillary microsample analytes include mass spectrometry (e.g., triple quadrupole mass spectrometry (TQMS), quadrupole time-of-flight (QTOF) mass spectrometry, or the like), chromatography (e.g., high-performance liquid chromatography (HPLC)), enzyme-linked immunosorbent assay (ELISA), electrophoresis, and/or the like. In certain embodiments, assessing the test mixture for one or more capillary microsample analytes is by triple quadrupole mass spectrometry (TQMS). A TQMS mass spectrometer is a tandem mass spectrometer consisting of two quadrupole mass analyzers in series, with a (non-mass-resolving) radio frequency (RF)-only quadrupole between them to act as a cell for collision-induced dissociation. This configuration is often abbreviated QqQ, or Q1q2Q3. TQMS mass spectrometers and details regarding the operation of same are available, a non-limiting example of which is the LCMS-8060NX Triple Quadrupole Liquid Chromatograph Mass Spectrometer (LC-MS/MS) available from Shimadzu. According to some embodiments, assessing the test mixture for one or more capillary microsample analytes is by quadrupole time-of-flight (QTOF) mass spectrometry. QTOF mass spectrometry is an analytical technique that advantageously combines the benefits of two different mass analyzers. Utilizing the high compound fragmentation efficiency of quadrupole technology in combination with the rapid analysis speed and high mass resolution capability of time-of-flight. QTOF MS instrumentation closely resembles that of a triple-quadrupole mass spectrometer, though the third quadrupole has been replaced by a time-of-flight tube. The first quadrupole (Q1) is capable of operating as a mass filter for the selection of specific ions based on their mass-to-charge ratio (m/z), or in radio frequency (RF) only mode where all ions are transmitted through the quadrupole. The second quadrupole (Q2) acts as a collision cell where ions are bombarded by neutral gas molecules such as nitrogen or argon, resulting in fragmentation of the ions by a process known as collision induced dissociation (CID). The Q2 can also act in RF-only mode without subsequent fragmentation of ions. After leaving the quadrupole, ions are reaccelerated into the ion modulator region of the time-of-flight analyzer where they are pulsed by an electric field and accelerated orthogonally to their original direction. QTOF mass spectrometers and details regarding the operation of same are available, a non-limiting example of which is the 6530 Q-TOF LC/MS available from Agilent.

Subjects

The types of subjects from whom the capillary microsample is obtained may vary. Generally such subjects are “mammals” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys).

In some embodiments, the subject is a human. Human subjects of interest include newborns (e.g., humans from birth to about 2 months of age), infants (newborn up to 1 year old), toddlers (1 year old up to 3 years old), preschoolers (3 years old up to 4 years old), children in middle childhood (4 years old up to 11 years old, such as 6 years old up to 8 years old, or 8 years old up to 11 years old), young teens (11 years old up to 14 years old) and teenagers (14 years old up to 18 years old). When the subject is a human adult, the subject may be a younger adult (an 18-30 year-old adult, e.g., a 21-28 year-old adult)), a middle-age adult (a 31-49 year-old adult), or an older adult (a 50 year-old or older adult (e.g., a 60-85 year-old elderly adult)). According to some embodiments, the human subject is a pregnant female. For example, the subject may be a human female in the first, second or third trimester of pregnancy.

Test Mixtures and Methods of Producing Same

As summarized above, the test mixture comprises the capillary microsample diluted into a stabilizing buffer comprising isopropanol, ethanol, methanol, or a combination thereof. In certain embodiments, the test mixture is a 1/5 to 1/20 dilution (e.g., a 1/8 to 1/12 dilution) of the capillary microsample into the stabilizing buffer. For example, according to some embodiments, the test mixture is 1/9 to 1/11 dilution (e.g., about a 1/10 dilution) of the capillary microsample into the stabilizing buffer. Any of the dilutions may comprise a capillary microsample of from 1 μL to 80 μL (e.g., 1 μL to 50 μL), 3 μL to 80 μL, 3 μL to 70 μL, 3 μL to 60 μL, 3 μL to 50 μL, 3 μL to 40 μL, 3 μL to 30 μL, 3 μL to 25 μL, 3 μL to 20 μL, 3 μL to 15 μL, 3 μL to 13 μL, 4 μL to 12 μL, 5 μL to 11 μL, 6 μL to 10 μL, or 7 μL to 9 μL, e.g., about 8 μL obtained from the subject diluted into the stabilizing buffer. In some embodiments, the entire capillary microsample obtained from the subject is diluted into the stabilizing buffer, where the entire capillary microsample obtained from the subject is 1 μL to 80 μL (e.g., 1 μL to 50 μL), 3 μL to 80 μL, 3 μL to 70 μL, 3 μL to 60 μL, 3 μL to 50 μL, 3 μL to 40 μL, 3 μL to 30 μL, 3 μL to 25 μL, 3 μL to 20 μL, 3 μL to 15 μL, 3 μL to 13 μL, 4 μL to 12 μL, 5 μL to 11 μL, 6 μL to 10 μL, or 7 μL to 9 μL, e.g., about 8 μL of capillary blood.

According to some embodiments, the methods of the present disclosure further comprise, prior to analyzing the test mixture, producing the test mixture by combining a 3 μL to 80 μL capillary microsample (e.g., 1 μL to 80 μL (e.g., 1 μL to 50 μL), 3 μL to 80 μL, 3 μL to 70 μL, 3 μL to 60 μL, 3 μL to 50 μL, 3 μL to 40 μL, 3 μL to 30 μL, 3 μL to 25 μL, 3 μL to 20 μL, 3 μL to 15 μL, 3 μL to 13 μL, 4 μL to 12 μL, 5 μL to 11 μL, 6 μL to 10 μL, or 7 μL to 9 μL, e.g., about 8 μL) obtained from the subject with the stabilizing buffer. The combining may comprise diluting the capillary microsample into the stabilizing buffer at a dilution of from 1/5 to 1/20. In certain embodiments, the combining comprises diluting the capillary microsample into the stabilizing buffer at a dilution of 1/8 to 1/12, e.g., 1/9 to 1/11, such as about 1/10.

The stabilizing buffer comprises isopropanol, ethanol, methanol, or combination thereof. According to some embodiments, the stabilizing buffer comprises the isopropanol, ethanol, methanol, or combination thereof, at a concentration of from 10% to 40%, 15% to 35%, or 20% to 30%, e.g., about 25%. In certain embodiments, the stabilizing buffer comprises, consists essentially of, or consists of the isopropanol, ethanol, methanol, or combination thereof (e.g., at a concentration of from 10% to 40%, 15% to 35%, or 20% to 30%, e.g., about 25%), and water. According to some embodiments, the stabilizing buffer comprises, consists essentially of, or consists of isopropanol (e.g., at a concentration of from 10% to 40%, 15% to 35%, or 20% to 30%, e.g., about 25%) and water. In certain embodiments, the stabilizing buffer consists of isopropanol (e.g., at a concentration of from 10% to 40%, 15% to 35%, or 20% to 30%, e.g., about 25%) and water.

According to some embodiments, the test mixture remains in liquid form upon its production (e.g., upon dilution of the capillary microsample into the stabilizing buffer) and at least until its assessment for one or more capillary microsample analytes. For example, in certain embodiments, the test mixture is never contacted with (e.g., adsorbed onto) a membrane (e.g., paper) upon its production and at least until its assessment for one or more capillary microsample analytes. By way of example, the test mixture may remain in liquid form upon its production and during any subsequent transportation and/or storage period prior to its assessment for one or more capillary microsample analytes.

Methods of Determining One or More Alleles of a Subject

Aspects of the present disclosure further include methods of determining one or more alleles of a subject. In certain embodiments, such methods comprise obtaining a 1 μL to 80 μL (e.g., 1 μL to 50 μL), 3 μL to 80 μL, 3 μL to 70 μL, 3 μL to 60 μL, 3 μL to 50 μL, 3 μL to 40 μL, 3 μL to 30 μL, 3 μL to 25 μL, 3 μL to 20 μL, 3 μL to 15 μL, 3 μL to 13 μL, 4 μL to 12 μL, 5 μL to 11 μL, 6 μL to 10 μL, or 7 μL to 9 μL (e.g., about 8 μL) capillary microsample from the subject, and diluting the capillary microsample into a stabilizing buffer comprising isopropanol, ethanol, methanol, or a combination thereof, to produce a test mixture. Such methods may further comprise purifying capillary microsample nucleic acids from the test mixture. According to some embodiments, such methods further comprise analyzing the purified capillary microsample nucleic acids to determine one or more alleles of a subject. Determining one or more alleles of a subject may comprise genotyping the subject, including but not limited to by any of the genotyping embodiments described elsewhere herein.

Obtaining the capillary microsample from the subject comprises pinpricking the subject and collecting the capillary microsample (1 μL to 80 μL (e.g., 1 μL to 50 μL), 3 μL to 80 μL, 3 μL to 70 μL, 3 μL to 60 μL, 3 μL to 50 μL, 3 μL to 40 μL, 3 μL to 30 μL, 3 μL to 25 μL, 3 μL to 20 μL, 3 μL to 15 μL, 3 μL to 13 μL, 4 μL to 12 μL, 5 μL to 11 μL, 6 μL to 10 μL, or 7 μL to 9 μL (e.g., about 8 μL)) from the site of the pinprick using a capillary tube. According to some embodiments, obtaining the capillary microsample from the subject comprises pinpricking a finger of the subject.

In certain embodiments, diluting the capillary microsample into a stabilizing buffer comprises placing a capillary tube comprising the capillary microsample into a container comprising the stabilizing buffer, and agitating the container to release and dilute the capillary microsample into the stabilizing buffer. The agitating may be by shaking, vortexing, and/or any other suitable approach. In some embodiments, the entire capillary microsample obtained from the subject is diluted into the stabilizing buffer, where the entire capillary microsample obtained from the subject is 1 μL to 80 μL (e.g., 1 μL to 50 μL), 3 μL to 80 μL, 3 μL to 70 μL, 3 μL to 60 μL, 3 μL to 50 μL, 3 μL to 40 μL, 3 μL to 30 μL, 3 μL to 25 μL, 3 μL to 20 μL, 3 μL to 15 μL, 3 μL to 13 μL, 4 μL to 12 μL, 5 μL to 11 μL, 6 μL to 10 μL, or 7 μL to 9 μL (e.g., about 8 μL) of capillary blood.

The diluting may comprise diluting the capillary microsample into the stabilizing buffer at a dilution of from 1/5 to 1/20. In certain embodiments, the diluting comprises diluting the capillary microsample into the stabilizing buffer at a dilution of 1/8 to 1/12, e.g., 1/9 to 1/11, such as about 1/10.

The stabilizing buffer comprises isopropanol, ethanol, methanol, or combination thereof. According to some embodiments, the stabilizing buffer comprises the isopropanol, ethanol, methanol, or combination thereof, at a concentration of from 10% to 40%, 15% to 35%, or 20% to 30%, e.g., about 25%. In certain embodiments, the stabilizing buffer comprises, consists essentially of, or consists of the isopropanol, ethanol, methanol, or combination thereof (e.g., at a concentration of from 10% to 40%, 15% to 35%, or 20% to 30%, e.g., about 25%), and water.

According to some embodiments, the stabilizing buffer comprises, consists essentially of, or consists of isopropanol (e.g., at a concentration of from 10% to 40%, 15% to 35%, or 20% to 30%, e.g., about 25%) and water. In certain embodiments, the stabilizing buffer consists of isopropanol (e.g., at a concentration of from 10% to 40%, 15% to 35%, or 20% to 30%, e.g., about 25%) and water.

According to some embodiments, the test mixture remains in liquid form upon its production (e.g., upon dilution of the capillary microsample into the stabilizing buffer) and at least until its assessment for one or more capillary microsample analytes. For example, in certain embodiments, the test mixture is never contacted with (e.g., adsorbed onto) a membrane (e.g., paper) from its production and at least until its assessment for one or more capillary microsample analytes. By way of example, the test mixture may remain in liquid form upon its production and during any subsequent transportation and/or storage period prior to its assessment for one or more capillary microsample analytes.

Any of the nucleic acid analysis methods described elsewhere herein (e.g., amplification, qPCR, sequencing, etc.) may be performed in the methods of determining one or more alleles of the subject. Such approaches are incorporated but not reiterated herein for purposes of brevity. Moreover, any of the types of alleles described elsewhere herein (e.g., SNP alleles (e.g., MAF SNP alleles), VNTR alleles, indel alleles, STR alleles, gene alleles, combinations thereof, etc.) may be determined. Such alleles are incorporated but not reiterated herein for purposes of brevity. The subject from whom the capillary microsample is obtained may be any subject of interest, including any of the types of subjects described elsewhere herein.

### Kits

Aspects of the present disclosure further include kits. The kits find use in a variety of applications, including for performing any of the methods of the present disclosure. The kits of the present disclosure may include any desired combination of reagents, containers, etc. described elsewhere herein for, e.g., obtaining a capillary microsample, diluting the capillary microsample into a stabilizing buffer, purifying nucleic acids from the resulting test mixture, and/or analyzing the nucleic acids (e.g., by qPCR or other desired approach).

In some embodiments, a kit of the present disclosure comprises a capillary tube, a stabilizing buffer comprising isopropanol, ethanol, methanol, or combination thereof, and nucleic acid analysis reagents.

According to some embodiments, the internal volume of a capillary tube provided in the kit is 3 μL to 80 μL, 3 μL to 70 μL, 3 μL to 60 μL, 3 μL to 50 μL, 3 μL to 40 μL, 3 μL to 30 μL, 3 μL to 25 μL, 3 μL to 20 μL, 3 μL to 15 μL, 3 μL to 13 μL, 4 μL to 12 μL, 5 μL to 11 μL, 6 μL to 10 μL, or 7 μL to 9 μL (e.g., about 8 μL). Such a capillary tube finds use in collecting a blood capillary microsample of the corresponding volume for diluting into the stabilizing buffer.

The stabilizing buffer comprises isopropanol, ethanol, methanol, or combination thereof. According to some embodiments, the stabilizing buffer comprises the isopropanol, ethanol, methanol, or combination thereof, at a concentration of from 10% to 40%, 15% to 35%, or 20% to 30%, e.g., about 25%. In certain embodiments, the stabilizing buffer comprises, consists essentially of, or consists of the isopropanol, ethanol, methanol, or combination thereof (e.g., at a concentration of from 10% to 40%, 15% to 35%, or 20% to 30%, e.g., about 25%), and water. According to some embodiments, the stabilizing buffer comprises, consists essentially of, or consists of isopropanol (e.g., at a concentration of from 10% to 40%, 15% to 35%, or 20% to 30%, e.g., about 25%) and water. In certain embodiments, the stabilizing buffer consists of isopropanol (e.g., at a concentration of from 10% to 40%, 15% to 35%, or 20% to 30%, e.g., about 25%) and water.

When the kit includes nucleic acid analysis reagents, in some embodiments, such reagents comprise genotyping primers (e.g., SNP genotyping primers, such as one or more MAF SNP genotyping primers). In certain embodiments, the kits find use in determining/confirming the identity of the subject from whom the capillary microsample was obtained.

Components of the kits may be present in separate containers, or multiple components may be present in a single container. For example, different nucleic acid analysis reagents (e.g., different genotyping primers or pairs thereof, and/or the like) may be provided in the same tube, or may be provided in different tubes.

In addition to the above-mentioned components, the kits of the present disclosure may further include instructions for using the components of the kit, e.g., to practice the methods of the present disclosure. According to some embodiments, the kits include instructions for obtaining a capillary microsample from a subject using the capillary tube, diluting the capillary microsample into the stabilizing buffer to form a test mixture, and purifying capillary microsample nucleic acids from the test mixture. The kits may further include instructions for analyzing the purified nucleic acids (e.g., to determine one or more alleles of the subject), e.g., by qPCR or the like. The instructions are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or sub-packaging), etc. In other embodiments, the instructions are present as an electronic storage data file present on a suitable computer readable storage medium, e.g., CD-ROM, diskette, Hard Disk Drive (HDD) etc. In yet other embodiments, the actual instructions are not present in the kit, but means for obtaining the instructions from a remote source, e.g., via the internet, are provided. An example of this embodiment is a kit that includes a web address where the instructions can be viewed and/or from which the instructions can be downloaded. As with the instructions, this means for obtaining the instructions is recorded on a suitable substrate.

Notwithstanding the appended claims, the present disclosure is also defined by the following embodiments:

1. A method of analyzing a capillary microsample obtained from a subject, the method comprising:
(i) assessing a test mixture for one or more capillary microsample analytes, wherein the test mixture comprises the capillary microsample diluted into a stabilizing buffer comprising isopropanol, ethanol, methanol, or a combination thereof; and
(ii) analyzing capillary microsample nucleic acids purified from the test mixture.

2. The method according to embodiment 1, wherein analyzing the capillary microsample nucleic acids comprises amplifying the capillary microsample nucleic acids.

3. The method according to embodiment 2, wherein analyzing the capillary microsample nucleic acids comprises quantitative polymerase chain reaction (qPCR).

4. The method according to any one of embodiments 1 to 3, wherein analyzing the capillary microsample nucleic acids comprises determining one or more alleles of the subject.

5. The method according to embodiment 4, wherein determining one or more alleles of the subject comprises genotyping the subject.

6. The method according to embodiment 4 or embodiment 5, wherein the one or more alleles comprises one or more polymorphic alleles.

7. The method according to embodiment 6, wherein the one or more polymorphic alleles comprises one or more single nucleotide polymorphism (SNP) alleles, one or more variable number of tandem repeat (VNTR) alleles, one or more insertion/deletion (indel) alleles, one or more short tandem repeat (STR) alleles, or any combination thereof.

8. The method according to any one of embodiments 4 to 7, wherein the one or more alleles comprises an allele of one or more genes.

9. The method according to embodiment 8, wherein the one or more genes comprise a gene that encodes a drug metabolizing enzyme, a gene that encodes a drug receptor, a gene that encodes a drug transporter, or any combination thereof.

10. The method according to any one of embodiments 1 to 9, wherein analyzing the capillary microsample nucleic acids comprises determining the presence or absence of a region of the Y-chromosome among genomic DNA present in the capillary microsample nucleic acids.

11. The method according to any one of embodiments 1 to 10, wherein assessing the test mixture for one or more capillary microsample analytes comprises determining the concentration of one or more capillary microsample analytes, if present, in the test mixture.

12. The method according to any one of embodiments 1 to 11, wherein assessing the test mixture for one or more capillary microsample analytes is by mass spectrometry.

13. The method according to embodiment 12, wherein the mass spectrometry is triple quadrupole mass spectrometry (TQMS).

14. The method according to embodiment 12, wherein the mass spectrometry is quadrupole time-of-flight (QTOF) mass spectrometry.

15. The method according to any one of embodiments 1 to 14, wherein the one or more capillary microsample analytes comprises one or more drugs of abuse.

16. The method according to embodiment 15, wherein the one or more drugs of abuse comprises a narcotic, an opioid, a stimulant, a depressant, a hallucinogen, a cannabinoid, or any combination thereof.

17. The method according to embodiment 15, wherein the one or more drugs of abuse comprises one or more opioids.

18. The method according to embodiment 17, wherein the one or more opioids comprises oxycodone, hydrocodone, morphine, methadone, fentanyl, heroin, or any combination thereof.

19. The method according to any one of embodiments 15 to 18, wherein the one or more drugs of abuse comprises gabapentin.

20. The method according to any one of embodiments 1 to 13, wherein the one or more capillary microsample analytes comprises one or more performance enhancing drugs.

21. The method according to embodiment 20, wherein the one or more performance enhancing drugs comprises an anabolic steroid, androstenedione, human growth hormone (HGH), erythropoietin (EPO), a diuretic, creatine, a stimulant, or any combination thereof.

22. The method according to embodiment 20 or embodiment 21, wherein the subject is a participant in an athletic contest.

23. The method according to any one of embodiments 1 to 14, wherein the one or more capillary microsample analytes comprises a therapeutic drug being administered to the subject, or a metabolite thereof.

24. The method according to embodiment 23, wherein the therapeutic drug is a drug for treatment of cancer, hypertension, epilepsy, hypercoagulation, or nausea.

25. The method according to any one of embodiments 1 to 24, wherein the method is performed iteratively on test mixtures comprising capillary microsamples obtained serially from the subject.

26. The method according to any one of embodiments 1 to 25, wherein the subject is a newborn.

27. The method according to any one of embodiments 1 to 25, wherein the subject is elderly.

28. The method according to any one of embodiments 1 to 27, wherein the test mixture is a 1/5 to 1/20 dilution of the capillary microsample into the stabilizing buffer.

29. The method according to any one of embodiments 1 to 27, wherein the test mixture is a 1/8 to 1/12 dilution of the capillary microsample into the stabilizing buffer.

30. The method according to embodiment 28 or embodiment 29, wherein the dilution comprises a capillary microsample of from 1 μL to 50 μL obtained from the subject diluted into the stabilizing buffer.

31. The method according to embodiment 28 or embodiment 29, wherein the dilution comprises a capillary microsample of about 6 μL to 10 μL obtained from the subject diluted into the stabilizing buffer.

32. The method according to any one of embodiments 1 to 30, further comprising, prior to analyzing the test mixture, producing the test mixture by combining a 1 μL to 50 μL capillary microsample obtained from the subject with the stabilizing buffer.

33. The method according to embodiment 32, wherein the combining comprises diluting the capillary microsample into the stabilizing buffer at a dilution of from 1/5 to 1/20.

34. The method according to embodiment 32, wherein the combining comprises diluting the capillary microsample into the stabilizing buffer at a dilution of from 1/8 to 1/12.

35. The method according to any one of embodiments 32 to 34, wherein the stabilizing buffer comprises the isopropanol, ethanol, methanol, or combination thereof, at a concentration of from 15% to 35%.

36. The method according to any one of embodiments 32 to 34, wherein the stabilizing buffer comprises the isopropanol, ethanol, methanol, or combination thereof, at a concentration of about 25%.

37. The method according to any one of embodiments 1 to 36, wherein the stabilizing buffer comprises, consists essentially of, or consists of isopropanol and water.

38. The method according to any one of embodiments 1 to 36, wherein the stabilizing buffer consists of isopropanol and water.

39. The method according to any one of embodiments 32 to 38, wherein the test mixture remains in liquid form upon its production and at least until step (i).

40. A method of determining one or more alleles of a subject, the method comprising:
obtaining a 1 μL to 50 μL capillary microsample from the subject;
diluting the capillary microsample into a stabilizing buffer comprising isopropanol, ethanol, methanol, or a combination thereof, to produce a test mixture;
purifying capillary microsample nucleic acids from the test mixture; and
analyzing the purified capillary microsample nucleic acids to determine one or more alleles of a subject.

41. The method according to embodiment 40, wherein obtaining the capillary microsample from the subject comprises pinpricking the subject and collecting the 1 μL to 50 μL capillary microsample from the site of the pinprick using a capillary tube.

42. The method according to embodiment 41, wherein obtaining the capillary microsample from the subject comprises pinpricking a finger of the subject.

43. The method according to any one of embodiments 40 to 42, wherein diluting the capillary microsample into a stabilizing buffer comprises placing a capillary tube comprising the 1 μL to 50 μL capillary microsample into a container comprising the stabilizing buffer, and agitating the container to release and dilute the capillary microsample into the stabilizing buffer.

44. The method according to any one of embodiments 40 to 43, wherein the diluting comprises diluting the capillary microsample into the stabilizing buffer at a dilution of from 1/5 to 1/20.

45. The method according to any one of embodiments 40 to 43, wherein the diluting comprises diluting the capillary microsample into the stabilizing buffer at a dilution of from 1/8 to 1/12.

46. The method according to any one of embodiments 40 to 45, wherein the test mixture remains in liquid form upon the diluting step until at least the purifying step.

47. The method according to any one of embodiments 40 to 46, wherein determining the one or more alleles comprises amplifying the purified capillary microsample nucleic acids.

48. The method according to embodiment 47, wherein determining the one or more alleles comprises quantitative polymerase chain reaction (qPCR).

49. The method according to any one of embodiments 40 to 48, wherein determining the one or more alleles of the subject comprises genotyping the subject.

50. The method according to embodiment any one of embodiments 40 to 49, wherein the one or more alleles comprises one or more polymorphic alleles.

51. The method according to embodiment 50, wherein the one or more polymorphic alleles comprises one or more single nucleotide polymorphism (SNP) alleles, one or more variable number of tandem repeat (VNTR) alleles, one or more insertion/deletion (indel) alleles, one or more short tandem repeat (STR) alleles, or any combination thereof.

52. The method according to any one of embodiments 40 to 51, wherein the one or more alleles comprises an allele of one or more genes.

53. The method according to embodiment 52, wherein the one or more genes comprise a gene that encodes a drug metabolizing enzyme, a gene that encodes a drug receptor, a gene that encodes a drug transporter, or any combination thereof.

54. The method according to any one of embodiments 40 to 53, wherein the analyzing comprises determining the presence or absence of a region of the Y-chromosome among genomic DNA present in the capillary microsample nucleic acids.

55. The method according to any one of embodiments 40 to 54, wherein the stabilizing buffer comprises the isopropanol, ethanol, methanol, or combination thereof, at a concentration of from 15% to 35%.

56. The method according to any one of embodiments 40 to 54, wherein the stabilizing buffer comprises the isopropanol, ethanol, methanol, or combination thereof, at a concentration of about 25%.

57. The method according to any one of embodiments 40 to 56, wherein the stabilizing buffer comprises, consists essentially of, or consists of isopropanol and water.

58. The method according to any one of embodiments 40 to 56, wherein the stabilizing buffer consists of isopropanol and water.

59. A kit comprising:
a capillary tube;
a stabilizing buffer comprising isopropanol, ethanol, methanol, or combination thereof; and
nucleic acid analysis reagents.

60. The kit of embodiment 59, further comprising instructions for obtaining a capillary microsample from a subject using the capillary tube, diluting the capillary microsample into the stabilizing buffer to form a test mixture, and purifying capillary microsample nucleic acids from the test mixture.

61. The kit of embodiment 59 or embodiment 60, wherein the capillary tube is a 1 μL to 50 μL capillary tube.

62. The kit of any one of embodiments 59 to 61, wherein the stabilizing buffer comprises the isopropanol, ethanol, methanol, or combination thereof, at a concentration of from 15% to 35%.

63. The kit of any one of embodiments 59 to 61, wherein the stabilizing buffer comprises the isopropanol, ethanol, methanol, or combination thereof, at a concentration of about 25%.

64. The kit of any one of embodiments 59 to 63, wherein the nucleic acid analysis reagents comprise genotyping primers.

65. The kit of embodiment 64, wherein the genotyping primers comprise SNP genotyping primers.

The following examples are offered by way of illustration and not by way of limitation.

### EXPERIMENTAL

### Example 1—Donor Identification of Capillary Microsamples by Genotyping

A modified capillary microsampling method (sometimes referred to herein as “isopropanol capillary microsampling” or “iso-CMS”) was recently developed. This method involves obtaining a capillary microsample of from about 6 μL to 10 μL from a subject and diluting the sample into a solution comprising isopropanol—a solvent safe for use in clinical settings. Iso-CMS enables highly accurate analyses of drug levels (including opiates) in blood to be performed on 8-ul blood samples obtained by a finger stick. The use of an iso-CMS-based opiate analysis method (iCOAM) would eliminate the problems associated with urinary drug testing for opioids (UDTO): the blood sample is directly obtained via a finger stick from a subject that has properly identified him/herself, and an authorized agent places it into the stabilizing buffer. This eliminates the chance for sample substitution or alteration. However, the utility of iCOAM (as well as a variety of other illicit and therapeutic drug analyses) would be markedly increased if the same blood sample obtained from a donor could also be analyzed to confirm donor identity. Of note, an iso-CMS sample is placed into a stabilizing buffer that contains isopropanol (e.g., about 25% isopropanol), and isopropanol is a solvent that is commonly used for many different analytical purposes. Of importance, isopropanol is the preferred alcohol that is used to isolate DNA for genomic analyses because: a smaller volume of isopropanol is required for DNA precipitation than with other alcohols (e.g., ethanol), and an isopropanol DNA precipitation can be performed at room temperature. Thus, DNA could be obtained from an iso-CMS sample, which would make it possible that the same sample that is used for measuring a drug level could be genotyped, thereby providing allelic information about the sample donor.

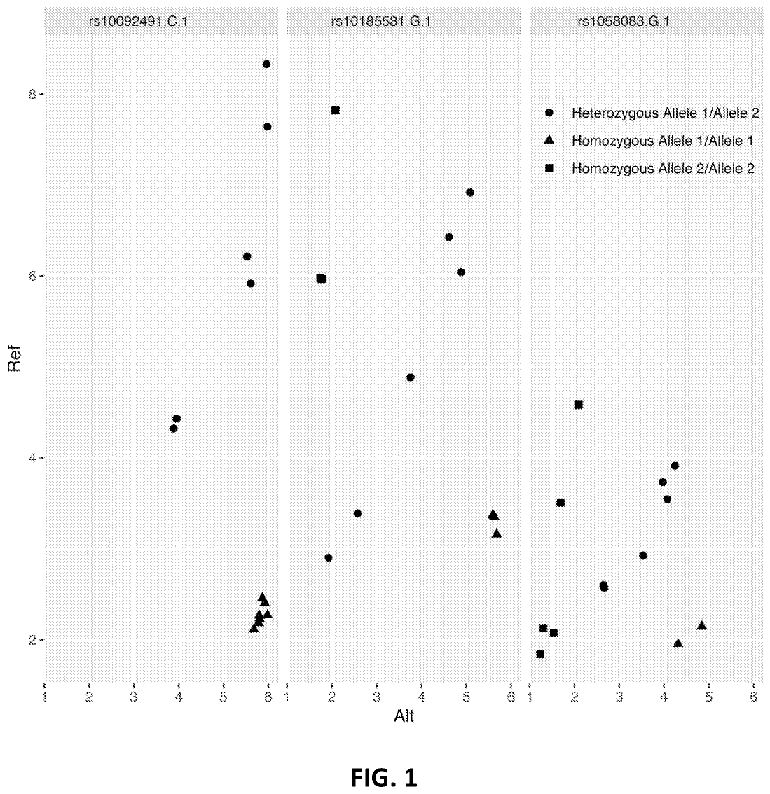

The present study sought to determine whether an iso-CMS sample could be used for donor identification. Twelve 8 μl blood capillary microsamples were obtained by pinprick, and then diluted into an isopropanol-containing stabilizing buffer (in this example, diluted 10-fold into a stabilizing buffer consisting of 25% isopropanol in water). DNA was prepared from the diluted iso-CMS sample using the Qiagen AllPrep™ DNA/RNA Mini Kit according to the manufacturer's procedures (Qiagen, Germantown, MD Cat #80204), and DNA was eluted from the column into a 50 μl volume of TE buffer. Then 4% (2 μl) of the eluted volume of sample was used for qPCR allele-specific genotyping. As proof of concept, the alleles at 3 SNP sites were measured: UBAC2 rs1058083 (reference and alternative alleles are A/G); FZD3 rs10092491 (T/C); and UXS1 rs10185531 (T/C). qPCR was performed using the IDT rhAmp SNP Assay (Integrated DNA Technologies, Coralville, Iowa) with specific sets of oligonucleotide primers designed by the manufacturer. The alleles were un-equivocally called for 35 of the 36 genotyping reactions (FIG. 1, Table 1).

TABLE 1
iso-CMS Genotyping Results

| Sample | rs1058083 | rs10092491 | rs10185531 |
|---|---|---|---|
| (Ref/Alt): | (A/G) | (T/C) | (T/C) |
| 1 | G/G | T/C | T/C |
| 2 | A/G | C/C | C/C |
| 3 | A/G | C/C | T/C |
| 4 | A/A | C/C | C/C |
| 5 | A/G | C/C | T/C |
| 6 | G/G | T/C | T/T |
| 7 | A/G | C/C | T/T |
| 8 | G/G | C/C | T/C |
| 9 | A/G | T/C | ND |
| 10 | A/A | T/C | T/T |
| 11 | A/G | T/C | T/T |
| 12 | A/A | C/C | T/C |

* Twelve iso-CMS samples were genotyped by qPCR at 3 SNP sites. The reference and alternative alleles at each SNP are shown in the top row, and donor genotypes at each SNP are indicated in the table. ND, genotype not determined. Although though only 3 SNPs were genotyped, each donor could be distinguished by their genotype.

Of importance, although only a limited number of SNP sites were investigated in this proof of concept experiment, each of the 12 donors could be distinguished based upon this allelic information.

As such, the present example demonstrates that genotyping can be performed on iso-CMS samples, which are 8 μl blood samples obtained from the site of a pinprick. The results establish that identifying allelic information can be obtained using a small aliquot of an iso-CMS sample, which can simultaneously be used for measurement of the blood levels of analytes such as illicit drugs, therapeutic drugs, performance enhancing drugs, and/or the like. Of note, the number of different genotypic tests that can performed on iso-CMS samples can be increased beyond that of the present experiments. For example, PCR-based methods for donor sex determination have long been established. In brief, the presence of PCR amplicons derived from the male Y-chromosome indicates male sex, while their absence indicates female sex. Therefore, established PCR amplification methods for male-specific genes (e.g., SRY, AMEL, and/or the like) can be used for donor sex determination in iso-CMS samples. Also, to further ensure that iso-CMS sample donors can be distinguished by the genotypic data, qPCR genotyping can be performed at many additional SNP sites. Of the 4-5 million human SNPs that have been identified, the SNPs selected for genotyping may be based upon their minor allele frequency (MAF) in the population, which is obtained from the NCBI dbSNP database (www.ncbi.nlm.nih.gov/snp/). SNPs with MAFs ranging from 25 to 48% may be selected because they have the most discriminatory power. Of importance, genotyping from only a limited number of SNP sites is required for donor identification since establishing the unique identify of a donor within the entire US population is not required. In contrast, all that would be required is to establish that the sample donor is the same as the one that provided prior samples, which can be when donor identity was confirmed using government issued identification (drivers' license, passport, etc.).

The ability to simultaneously measure drug levels and genotypes from a single micro-volume blood sample obtained via a minimally invasive procedure will also transform the ability to optimize drug dosing, especially in populations that have been difficult to study. Little is known about how a drug will behave in humans prior to clinical testing since most of the pre-clinical information about a drug is obtained from animal and in vitro human cellular studies, which are not very predictive of what will happen in humans. Clinical studies involve a relatively small number of subjects, and even for the drugs that receive regulatory approval and are marketed, there is often very little information about how genetic variation can alter their metabolism. While it is known that pharmacogenetic factors have a strong effect on drug metabolism and on the incidence of adverse drug reactions, the utilization of pharmacogenetic markers in clinical practice has been far below what was expected. Moreover, nothing is typically known about the metabolism of a drug in babies or in the elderly, which is at least partly due to the difficulties associated with obtaining the volume of blood that is required for measurement of drug concentrations with conventional methods. Hence, the present methods will greatly increase the ability to perform pharmacogenetic studies and transform the present understanding of drug metabolism.

Methods

UBAC2 (Hs.GT.rs1058083.G.1) Reference: GGTTAATTTTGCTCA GAGTAT CCArGAGTT (SEQ ID NO:1)/GT4 Alternative: GGTTAATTTTGCTCAGAGTATCCGrGAGTT (SEQ ID NO:2)/GT4 Reverse: GCAGGCAAGGGATCGTTTCTCrCTCTT (SEQ ID NO:3)/GT3. FZD3 (Hs.GT.rs10092491.C.1): Reference: TCCAGATAGAGCTAAAACTGAAGTrUTTCC (SEQ ID NO:4)/GT1 Alternative: TCCAGATAGAGCTAAAACT GAAGCrUTTCC (SEQ ID NO:5)/GT1 Reverse: GCAACCAGTATCCCCGCAAArCTAAC (SEQ ID NO:6)/GT1. UXS1 (Hs.GT.rs10185531.G.1): Reference: CCGTCATTCTCTCAGTTCTTATrCTCTG (SEQ ID NO:7)/GT4

Alternative: CCGTCATTCTCTCAGTTCTTACrCTCTG (SEQ ID NO:8)/GT4 Reverse: GCCTTCTCTGCACAGT TCATTGGrCTCCT (SEQ ID NO:9)/GT2. Reference and alternative alleles are indicated in bold. qPCR genotyping was performed using the Quantstudio 3 instrument with the IDT rhAmp Genotyping Master Mix (Cat #1076015) and Reporter Mix Kit (Cat #1076021) according to the manufacturer's protocols. The FAM and VIC dyes were used for genotyping the reference and alternative alleles, respectively. Allele calls were made by the instruments genotyping software.

### Example 2—Capillary Microsample Analyte Assessment

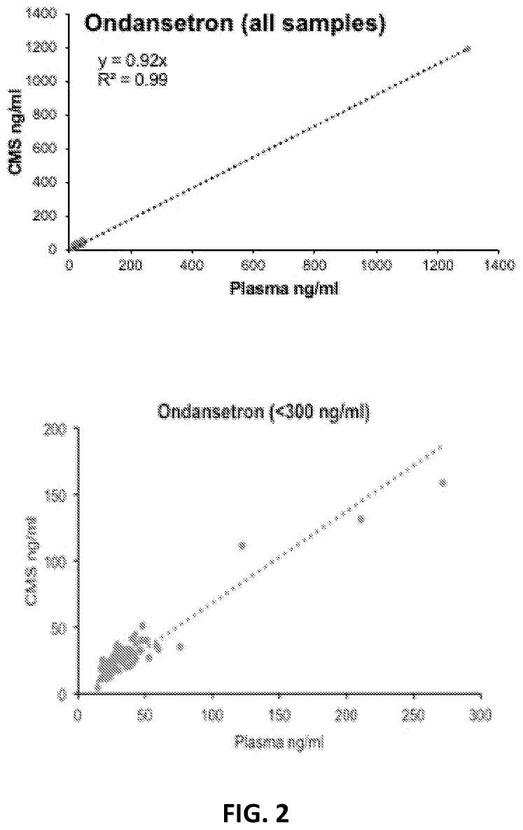

The current gold standard in clinical practice is measurement of drug levels in venous plasma. In this example, iso-CMS performance in a clinical setting was assessed by simultaneously obtaining venipuncture and iso-CMS samples after ondansetron was administered to 44 surgical patients (n=60 samples total due to sample collection at various times after ondansetron administration). The [ONDS] were determined by LCMS analysis using our published method, which was successfully used to analyze 372 blood samples in our pharmacokinetic study. The R² comparing the plasma and iso-CMS measurements was 0.99, which indicates that iso-CMS accurately measured the [ONDS] in a clinical setting (FIG. 2). The results of other statistical tests (Spearman Rho, Pearson's correlation) confirmed that there was a very strong concordance between the two measurements (Table 2).

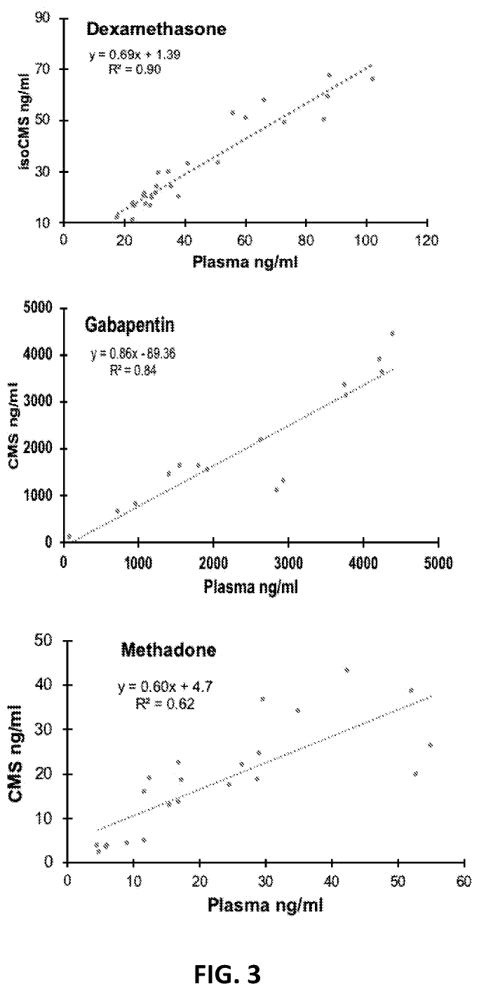

Also determined was whether iso-CMS could measure the concentrations of three other drugs in samples obtained in a clinical setting. Two are commonly used in neurosurgical patients (gabapentin and dexamethasone), and methadone is a commonly used opioid. While gabapentin (Neurontin) is used for treating epilepsy and postherpetic neuralgia, 90% of its use is for off-label treatment of chronic pain, insomnia, drug/alcohol addiction, anxiety, bipolar disorder, menopausal conditions, and other disorders. Since opioid abusers report that gabapentin potentiates an opioid ‘high’, 40-65% of individuals with gabapentin prescriptions misuse it for recreational purposes. Therefore, screening for illicit gabapentin ingestion is an indicator for possible drug abuse. Plasma and iso-CMS samples were simultaneously obtained from 29, 15 or 22 subjects receiving dexamethasone, gabapentin or methadone, respectively. Drug concentrations were measured by LCMS analysis. The amounts of each of these drugs could be independently determined in each sample based upon their distinct retention time and m/z parameters. Statistical analyses (R², Pearson correlation, Spearman Rho) indicated that there was strong concordance between the iso-CMS and plasma measurements for all three drugs (FIG. 3, Table 2).

TABLE 2
iso-CMS Genotyping Results

| Drug | N | R² | Pearson | Spearman |
|---|---|---|---|---|
| Gabapentin | 15 | 0.83 | 0.92 | 0.85 |
| Methadone | 22 | 0.62 | 0.99 | 0.90 |
| Dexameth | 29 | 0.90 | 0.95 | 0.93 |
| Ondansetron | 60 | 0.99 | 0.99 | 0.82 |

* This table shows the number of samples, and the statistics comparing the measured iso-CMS and plasma levels for each drug using the R2, Pearson's correlation and Spearman Rho tests.

Methods

Eight μL of plasma was placed in an 8-uL EDTA-coated glass capillary tube (Vitrex Medical A/S, Denmark). The capillary tube was placed within a centrifuge tube with 72-μL of a solution that consisted of 25% isopropanol in water at the clinical site. The mixture was vortexed thoroughly, and subsequent dilutions were prepared from the 1/10 diluted plasma sample. Three volumes of ice-cold acetonitrile were added, and proteins were precipitated, and the mixture was centrifuged. The supernatant was evaporated to dryness, and then re-suspended in HPLC water. The samples were analyzed on an Agilent QTOF 6520 equipped with an Infinity 1290 UPLC system. Drug concentrations were calculated relative to those of a standard curve using Agilent QTOF Quantitative Analysis software.

Accordingly, the preceding merely illustrates the principles of the present disclosure. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein.

What is claimed is:

1. A method of analyzing a capillary microsample obtained from a subject, the method comprising:
(i) assessing a test mixture for one or more capillary microsample analytes, wherein the test mixture comprises the capillary microsample diluted into a stabilizing buffer comprising isopropanol, ethanol, methanol, or a combination thereof; and
(ii) analyzing capillary microsample nucleic acids purified from the test mixture.

2. The method according to claim 1, wherein analyzing the capillary microsample nucleic acids comprises determining one or more alleles of the subject.

3. The method according to claim 2, wherein determining one or more alleles of the subject comprises genotyping the subject.

4. The method according to claim 2 or claim 3, wherein the one or more alleles comprises one or more polymorphic alleles.

5. The method according to claim 4, wherein the one or more polymorphic alleles comprises one or more single nucleotide polymorphism (SNP) alleles, one or more variable number of tandem repeat (VNTR) alleles, one or more insertion/deletion (indel) alleles, one or more short tandem repeat (STR) alleles, or any combination thereof.

6. The method according to any one of claims 2 to 5, wherein the one or more alleles comprises an allele of one or more genes.

7. The method according to any one of claims 1 to 6, wherein assessing the test mixture for one or more capillary microsample analytes comprises determining the concentration of one or more capillary microsample analytes, if present, in the test mixture.

8. The method according to any one of claims 1 to 7, wherein the one or more capillary microsample analytes comprises one or more drugs of abuse.

9. The method according to claim 8, wherein the one or more drugs of abuse comprises a narcotic, an opioid, a stimulant, a depressant, a hallucinogen, a cannabinoid, or any combination thereof.

10. The method according to any one of claims 1 to 9, wherein the one or more capillary microsample analytes comprises one or more performance enhancing drugs.

11. The method according to any one of claims 1 to 10, wherein the one or more capillary microsample analytes comprises a therapeutic drug being administered to the subject, or a metabolite thereof.

12. The method according to any one of claims 1 to 11, wherein the method is performed iteratively on test mixtures comprising capillary microsamples obtained serially from the subject.

13. The method according to any one of claims 1 to 12, wherein the test mixture is a 1/5 to 1/20 dilution of the capillary microsample into the stabilizing buffer.

14. The method according to claim 13, wherein the dilution comprises a capillary microsample of from 1 μL to 50 μL obtained from the subject diluted into the stabilizing buffer.

15. The method according to any one of claims 1 to 14, further comprising, prior to analyzing the test mixture, producing the test mixture by combining a 1 μL to 50 μL capillary microsample obtained from the subject with the stabilizing buffer.

16. The method according to claim 15, wherein the stabilizing buffer comprises the isopropanol, ethanol, methanol, or combination thereof, at a concentration of from 15% to 35%.

17. The method according to any one of claims 1 to 16, wherein the stabilizing buffer comprises, consists essentially of, or consists of isopropanol and water.

18. A method of determining one or more alleles of a subject, the method comprising:
obtaining a 1 μL to 50 μL capillary microsample from the subject;
diluting the capillary microsample into a stabilizing buffer comprising isopropanol, ethanol, methanol, or a combination thereof, to produce a test mixture;
purifying capillary microsample nucleic acids from the test mixture; and
analyzing the purified capillary microsample nucleic acids to determine one or more alleles of a subject.

19. The method according to claim 18, wherein obtaining the capillary microsample from the subject comprises pinpricking the subject and collecting the 1 μL to 50 μL capillary microsample from the site of the pinprick using a capillary tube.

20. A kit comprising:
a capillary tube;
a stabilizing buffer comprising isopropanol, ethanol, methanol, or combination thereof; and
nucleic acid analysis reagents.
